SEQUENCE LISTING (26 sequences)

SEQ ID NO: 1 - DNA, 28 bp, Artificial
  Synthesized nucleic acids
aaaagctatagattgctcataacttcat

SEQ ID NO: 2 - DNA, 28 bp, Artificial
  Synthesized nucleic acids
atgaagttatgagcaatctatagctttt

SEQ ID NO: 3 - DNA, 30 bp, Artificial
  VapB-forward primer
ggaattccatatgagcatgatggctgaaag

SEQ ID NO: 4 - DNA, 28 bp, Artificial
  VapB-R-1 primer
ccgctcgagttaccactccttgcgtagc

SEQ ID NO: 5 - DNA, 25 bp, Artificial
  VapB-R-2 primer
ccgctcgagccactccttgcgtagc

SEQ ID NO: 6 - DNA, 29 bp, Artificial
  VapC forward primer
ggaattccatatgacctccggatctgcgc

SEQ ID NO: 7 - DNA, 28 bp, Artificial
  VapC reverse primer
ccgctcgagttattcagggtggatttcg

SEQ ID NO: 8 - DNA, 33 bp, Artificial
  D9A forward primer
ggatctgcgctttttgataccaatattcttatt

SEQ ID NO: 9 - DNA, 33 bp, Artificial
  D9A reverse primer
aataagaatattggtatcaaaaagcgcagatcc

SEQ ID NO: 10 - DNA, 33 bp, Artificial
  E43A forward primer
ctgattacctggatggaggtgatggttggcgct

SEQ ID NO: 11 - DNA, 33 bp, Artificial
  E43A reverse primer
agcgccaaccatcacctccatccaggtaatcag

SEQ ID NO: 12 - DNA, 33 bp, Artificial
  D90A forward primer
aagcttaagctgccggatgccatcattctggcg

SEQ ID NO: 13 - DNA, 34 bp, Artificial
  D90A reverse primer
cgccagaatgatggcatccggcagcttaagcttt

SEQ ID NO: 14 - DNA, 33 bp, Artificial
  D111A forward primer
acgcggaatacgaaggattttgccggtattcct

SEQ ID NO: 15 - DNA, 33 bp, Artificial
  D111A reverse primer
aggaataccggcaaaatccttcgtattccgcgt

SEQ ID NO: 16 - protein, 20 aa, Artificial
  N-terminal tag
MGSSHHHHHHSSGLVPRGSH

SEQ ID NO: 17 - protein, 8 aa, Artificial
  Synthesized peptide
GMEYQRQL

SEQ ID NO: 18 - protein, 8 aa, Artificial
  Synthesized peptide
GMEAQRQL

SEQ ID NO: 19 - protein, 8 aa, Artificial
  Synthesized peptide
GMEYARQL

SEQ ID NO: 20 - protein, 8 aa, Artificial
  Synthesized peptide
GMEAARQL

SEQ ID NO: 21 - protein, 9 aa, Artificial
  Synthesized peptide
GMEYQRQLR

SEQ ID NO: 22 - protein, 10 aa, Artificial
  Synthesized peptide
GMEYQRQLRK

SEQ ID NO: 23 - protein, 8 aa, Artificial
  Synthesized peptide
TNILIDLF

SEQ ID NO: 24 - protein, 9 aa, Artificial
  Synthesized peptide
DTNILIDLF

SEQ ID NO: 25 - protein, 9 aa, Artificial
  Synthesized peptide
TNILIDLFS

SEQ ID NO: 26 - protein, 10 aa, Artificial
  Synthesized peptide
DTNILIDLFS

(19) United States
(12) Patent Application Publication
(10) Pub. No.: US 2024/0101603 A1
(43) Pub. Date: Mar. 28, 2024

(54) ANTIBACTERIAL PEPTIDES AND COMPOUNDS TARGETING TOXIN-ANTITOXIN SYSTEM OF KLEBSIELLA PNEUMONIAE, AND THEIR USE

(71) Applicant: SEOUL NATIONAL UNIVERSITY R&DB FOUNDATION, Seoul (KR)

(72) Inventors: Sung Min KANG, Seoul (KR); Bong Jin LEE, Seoul (KR); Cheng Long JIN, Seoul (KR); Do Hee KIM, Jeju-do (KR)

(21) Appl. No.: 18/272,980

(22) PCT Filed: Jan. 20, 2022
(86) PCT No.: PCT/KR2022/001034

§ 371 Date: Jul. 18, 2023

(30) Foreign Application Priority Data
Jan. 20, 2021 (KR) 1020210007796

(51) Int. Cl. C07K 7/06; A61K 31/445; A61K 31/538; A61P 31/04

(52) U.S. Cl. CPC C07K 7/06; A61K 31/445; A61K 31/538; A61P 31/04; A61K 38/00

(57) ABSTRACT

The present invention relates to an antibacterial peptide and an antibacterial compound targeting the Klebsiella pneumoniae VapBC toxin-antitoxin system, or their use.

### CROSS-REFERENCE TO RELATED APPLICATIONS

This application is a U.S. National Phase Application of PCT/KR2022/001034, filed Jan. 20, 2022, which claims priority under 35 U.S.C. § 119 to KR 10-2021-0007796, filed Jan. 20, 2021, the contents of which applications are incorporated herein by reference in their entireties for all purposes.

### SEQUENCE LISTING STATEMENT

Incorporated herein by reference in its entirety is a Sequence Listing named “W117620017-SEQUENCE-LISTING”, which is being submitted to the USPTO via EFS-web on even date herewith as an ASCII text file 5 KB in size. This file, which was created on Jul. 7, 2023, constitutes both the paper and computer readable form of the Sequence Listing.

### BACKGROUND OF THE INVENTION

### 1. Field of the Invention

The present invention relates to an antibacterial peptide and an antibacterial compound, targeting the Klebsiella pneumoniae toxin-antitoxin system.

### 2. Description of the Related Art

Klebsiella pneumoniae is one of the most important opportunistic infectious agents. It is widespread in the environment, including water, solid phases, or leaf surfaces. Klebsiella pneumonia (K. pneumoniae) causes infection of the upper respiratory tract, urinary tract and bloodstream, and causes pneumonia. Currently, Klebsiella pneumoniae is considered a serious threat to the health of people around the world. This has an extremely plastic genome and tends to induce antibiotic resistance. In particular, drug-resistant Klebsiella pneumoniae, which is also resistant to carbapenem, is emerging as a global problem.

The overall biological functional system of toxin-antitoxin (TA) is very diverse. The initial role identified for the TA system was to maintain the plasmid. Soon after, similar functions of the TA system, such as postsegregational killing, were also observed. Furthermore, the TA system has been shown to mediate programmed cell death due to stresses such as antibiotic treatment, phage infection, DNA damage, oxidative stress, and high temperature. Recently, it has been shown that the TA system may play a role in the formation of bacterial persister cells.

Four main types of TA systems are classified according to the mechanism by which antitoxins inhibit toxins and the characteristics of antitoxins. Antitoxins of type I and type III TA systems are RNA molecules that regulate the level of toxins in cells. RNA antitoxins inhibit the translation of mRNA toxins (type I) and inhibit the activity of protein toxins by direct binding (type III). However, antitoxins of type II and type IV TA systems are proteins. These protein antitoxins inhibit the activity of toxins by binding directly to protein toxins (type II), or neutralize the toxic activity of toxins by causing the opposite effect on targets without binding directly (type IV). For type V and type VI TA systems, there is only a single case with different regulatory principles for each.

The bacterial type II TA system includes two proteins, a biophysically stable toxin and a relatively unstable antitoxin. In a normal state, the expression levels of toxins and antitoxins are well harmonized, so that the bacteria can survive normally because the antitoxins offset the harmful effects of the toxins. However, unstable antitoxins degrade more rapidly under stressful conditions, including antibiotic treatment, poor nutrition, and adverse temperatures. The toxin is then freed from the antitoxin, and the toxicity of the freed toxin damages the bacteria.

Type II TA systems are more widely distributed in pathogenic bacteria than in non-pathogenic bacteria. This suggests that the TA system is closely related to bacterial pathogenicity. In addition, the TA system is not found in any eukaryotic organism, so it is a promising drug target for bacterial pathogens. Dozens of structures of the TA system of pathogenic bacteria have been disclosed through structural and functional studies. However, structural information about the TA system of Klebsiella pneumoniae is still unknown. To gain molecular insight and validate the TA system of Klebsiella pneumoniae as a future drug target, it is important to investigate the structure of the TA system of Klebsiella pneumoniae.

VapBC is one of the largest families of Type II TA systems. To date, at least 11 complex structures have been identified in the VapBC TA system: Neisseria gonorrheae FitAB, Shigella exneri VapBC; Rickettsia felis VapBC2; Caulobacter crescentus VapC1; Haemophilus inuenza VapC1; and Mycobacterium tuberculosis VapBC3, VapBC5, VapBC11, VapBC15, VapC26, and VapBC30. In these VapBC complexes, VapB includes a variable DNA binding domain and a flexible region and is structurally very complicated while VapC represents a preserved rigid domain.

VapC toxins contain a pilus contractile protein (PilT) N-terminal (PIN) domain that is important for ribonuclease (RNase) activity. This PIN domain consists of a protein of approximately 130 amino acids and contains a strictly preserved negatively charged acid quartet of residues coordinated with Mg²⁺ at the active site. On the other hand, the homologous antitoxin VapB folds into a ribbon-helix-helix (RHH) DNA-binding motif at the N-terminus and wraps around the active site of VapC via the C-terminal helix. Because VapB sterically blocks the catalytic site of VapC and acts as a rigid binding inhibitor, molecules that inhibit the binding between VapB and VapC can produce and release free toxins. There are previous studies of these inhibitory molecules that act as binding competitors and result in growth arrest or eventual cell death in other pathogenic bacteria. The use of the TA system as an antibacterial strategy through artificial activation of toxins has been proposed as a potential approach to antibiotic therapy. In some recent studies, sequence-specific antisense peptide nucleic acid oligomers and peptide-based inhibitors have been shown to cause bacterial cell death.

### BRIEF SUMMARY OF THE INVENTION

It is an object of the present invention to provide an antibacterial peptide or an antibacterial compound, targeting the Klebsiella pneumoniae toxin-antitoxin system.

To achieve the above object, the present invention provides a novel peptide or compound.

Specifically, the present invention provides an antibacterial peptide that inhibits the binding of VapB and VapC in the Klebsiella pneumoniae toxin-antitoxin complex VapBC, and activates VapC toxin by destroying or inhibiting the binding of the Klebsiella pneumoniae toxin-antitoxin complex VapBC.

The present invention also provides a compound that inhibits the binding of VapB and VapC in the Klebsiella pneumoniae toxin-antitoxin complex VapBC, and inhibits the formation of the complex VapBC by blocking the interface pocket in the toxin-antitoxin complex VapBC at a low concentration.

In addition, the present invention provides an antibacterial composition, a quasi-drug, or an external preparation comprising the peptide or compound of the present invention.

### ADVANTAGEOUS EFFECT

The antibacterial peptide of the present invention mimics the binding interface of the Klebsiella pneumoniae toxin-antitoxin complex VapBC, which destroys the binding of the complex VapBC and artificially activates the toxin VapC, resulting in the death of Klebsiella pneumoniae by the RNase activity of the toxin. In addition, the small molecule compound of the present invention inhibits the binding of the complex VapBC by closing the interface pocket between VapB and VapC in the complex VapBC, but does not affect the RNase activity of the toxin VapC. Therefore, the antibacterial peptide and small molecule compound of the present invention can be used as an antibiotic composition that provides a novel strategy for controlling Klebsiella pneumoniae by using thereof as an inhibitor.

### BRIEF DESCRIPTION OF THE DRAWINGS

FIG. 1 shows the results of growth analysis and CFU measurement. The bacteria used for the validation of the Klebsiella pneumoniae VapBC TA system is E. coli. (A, B, and C) Each curve represents each variable shown in the table within the graph. Data represent mean values obtained from triplicate reactions. Standard deviations are indicated by error bars. (A) Growth analysis of the Klebsiella pneumoniae VapBC TA system. (B) CFU measurements of plates treated with 0.2% glucose (arabinose). (C) CFU measurements of plates treated with 0.2% glucose (arabinose) and 0.5 mM IPTG.

FIG. 2 shows the overall structural information of the Klebsiella pneumoniae VapBC (PDB ID: 7BY3). (A, B, and C) Subunits of the antitoxin VapB are shown in yellow and subunits of the toxin VapC are shown in light purple. (A) Heterotetrameric assembly of the VapBC complex containing intermolecular interfaces formed by dimerization of VapC. (B) Heterotetrameric assembly of the VapBC complex containing intermolecular interfaces formed by dimerization of the N-terminal DNA-binding domain of VapB. (C) Schematic diagram of the VapBC heterodimer showing the secondary structure architecture. (D) Secondary structure analysis of the N-terminal DNA-binding domain of VapB.

FIG. 3 shows the structural comparison of the Klebsiella pneumoniae VapBC and the homologues thereof. (A) Sequence alignment of the Klebsiella pneumoniae VapC and the homologues thereof. The secondary structure elements of these proteins are indicated above the sequence alignment. Extremely preserved residues are highlighted. Among them, the acidic active site residues and the remaining residues are indicated by triangles. (B) Electrostatic surface of VapC (PDB ID: 7BY3) (left). The acidic active sites of VapC are shown enlarged with cartoon and stick figures showing the positions of seven preserved residues in the structure of each homolog (right). (C) Comparison of VapBC complexes (PDB ID: 7BY3, 6A7V, 5X3T, 5K8J, and 6NKL in order). Each VapBC complex is shown in the same direction based on the VapC structure.

FIG. 4 shows the active site and RNase activity of VapC. (A) Two forms of VapBC complex structure: open form (left) (PDB ID: 7BY2) and closed form (right) (PDB ID: 7BY3). (B) Two forms of the active sites are shown along with a cartoon diagram of VapC. The four preserved residues are shown as stick figures. A 2mFo-DFc electron density map of the contour of the Mg²⁺ site at σ is also shown (calculated by PHENIX). In the open form VapC active site, the coordinated Mg²⁺ is located in the middle of the residues D9, E43, and D90. In the closed form VapC active site, R79 of VapB causes the displacement of Mg²⁺. Accordingly, R79 of VapB fills the original position of Mg²⁺, and Mg²⁺ moves to the middle of D9, D90, and D111. (C) In vitro RNase activity of VapC, VapC mutants, and VapBC. After treating the proteins with EDTA to remove metal ions, 10 mM Mg²⁺ was added to the proteins. In the analysis, 10 μM protein was used, and a general RNase inhibitor (40 units) was used to prevent unintentional contamination. The kinetics of the RNase activity of these proteins was recorded for 1 hour. The displayed data were obtained through three independent experiments.

FIG. 5 shows the heterodimer interface of the VapBC complex (PDB ID: 7BY3) and the complex disruption efficacy using the designed peptide. (A)-(B) Residues involved in the interaction network are indicated by bars. Schematic diagrams of hydrophilic and hydrophobic interactions are provided in the table. (A) Intermolecular interaction network of VapB α3 helix. (B) Intermolecular interaction network of VapC α1 helix. (C)-(D) RNase activity due to peptides added to the VapBC complex. The activity produced by the peptide is plotted relative to the activity of VapC. In this complex disruption assay, 100 μM peptide was added to 10 μM protein and the complex disruption efficacy was evaluated. Data are presented as the mean±SD of three independent experimental values. (C) Efficacy of the peptides designed based on the sequence of VapB α3 helix. (D) Efficacy of the peptides designed based on the sequence of VapC α1 helix.

FIG. 6 shows the effect of the peptides on free VapC. Data represent mean values obtained from triplicate reactions. Standard deviations are indicated as error bars. Free VapC was treated with the designed peptide of the present invention.

FIG. 7 shows the efficacy of the final compound represented by a surface pocket and molecular docking on VapC (PDB ID: 7BY3). (A) The surface of VapC, including a cartoon of VapB. The negatively charged active site cavity and the surface gap interface pocket are connected via the α2-α3 loop of VapB. The compound-binding site of VapC is located on the surface gap interface pocket denoted by the interface pocket valley. (B) RNase activity of the compound when the VapBC complex is added. The activity generated by the compound is plotted in proportion to the activity of VapC. The activity of the compound without the VapBC complex was also measured. In the complex disruption analysis, the compound (1, 3.16, and 10 μM) was added to 10 μM protein and the complex disruption efficacy was evaluated. The activity was measured based on the initial velocity during the first hour. Data are presented as the mean±SD of three independent experimental values.

FIG. 8 shows the effect of small molecules on free VapC. Data represent mean values obtained from triplicate reactions. Standard deviations are indicated by error bars. Free VapC was treated with the small molecule compound 1 and compound 2 of the present invention.

FIG. 9 shows the inhibition curves of (A) compound 1 and (B) compound 2 of the present invention. Data represent mean values obtained from triplicate reactions. Standard deviations are indicated by error bars.

FIG. 10 shows the molecular docking results of the compound in the VapC structure (PDB ID: 7BY3) including detailed interaction networks. In the cartoon diagram, the ligand-binding residues of VapC are shown as bars and labeled. A 2D ligand-VapC interaction diagram was generated using LigPlot⁺. The residues involved in HB are shown and HB is indicated by a dotted line. The residues that form non-binding contacts with the ligand are indicated by spoke arcs. (a) Interaction data for compound 1. (b) Interaction data for compound 2.

FIG. 11 shows the results of isothermal titration calorimetry (ITC) analysis. The ITC results were analyzed from combination parameters of (A) VapB & VapC, (B) VapC & Peptide VapB⁷¹⁻⁷⁸, (C) VapC & Peptide VapB^{71-78, Y74A}, (D) VapC & Peptide VapB^{71-78, Q75A} (E) VapC & Compound 1, (F) VapC & Compound 2, (G) VapB & DNA, and (H) VapBC & DNA. The sequences of DNA in (G) and (H) are represented by SEQ ID NO: 1 (forward: 5′-AAAAGCTATAGATTGCTCATAACTTCAT-3′) and SEQ ID NO: 2 (reverse: 5′-ATGAAGTTATGAGCAATCTATAGCTTTT-3′). The components participating in each experiment and the resulting combination parameters are shown in each graph.

### DESCRIPTION OF THE PREFERRED EMBODIMENTS

The present invention provides a peptide that disrupts or inhibits the binding between VapB and VapC in the toxin-antitoxin complex VapBC of Klebsiella pneumoniae.

The term “peptide” used herein includes polymers of amino acid residues linked together by peptide (amide) bonds, and refers to proteins, polypeptides, and peptides of any size, structure, or function. The peptide or polypeptide may refer to an individual protein, or a group of proteins.

The term “amino acid” used herein refers to a molecule containing an amino group and a carboxyl group. The amino acid includes an alpha-amino acid and a beta-amino acid. The amino acid can be a non-natural amino acid or a natural amino acid. Exemplary amino acids include natural alpha-amino acids such as D- and L-isomers of 20 common naturally occurring alpha amino acids found in peptides, non-natural alpha-amino acids, natural beta-amino acids (e.g., beta-alanine), and non-natural beta-amino acids, but not always limited thereto. The amino acids used in the construction of the peptide of the present invention may be prepared by organic synthesis, or may be obtained by other routes, such as isolation or decomposition from natural sources. The amino acid can be obtained commercially or synthesized.

Through the structure of the VapBC complex, the α3 helix of the antitoxin VapB and the al helix of the toxin VapC are involved in the binding between the toxin protein VapC and the antitoxin protein VapB. The peptide may disrupt or inhibit the binding of the α3 helix of the antitoxin VapB to the α1 helix of the toxin VapC, and may not affect the activity of the toxin protein VapC.

The peptide may have any one amino acid sequence selected from the group consisting of the sequences represented by SEQ ID NO: 16 to NO: 25. Amino acids in which one or several amino acids are added, substituted, or deleted may also be used as the peptide.

The present invention first provides a main crystal structure of the VapBC complex of Klebsiella pneumoniae at a resolution of 2.00 Å. In the complex structure analysis, the present inventors moved Mg²⁺ coordinated by R79 of VapB from D9, E43, and D90 of adjacent VapC to D9, D90, and D111 of VapC, and confirmed that the RNase activity of VapC was removed. In addition, it was further verified by site-induced mutagenesis and in vitro RNase activity experiments. The present inventors designed a peptide that mimics the binding interface of the VapBC complex, and confirmed that the peptide successfully induced the RNase activity of the VapC toxin, thereby disrupting the VapBC complex. In addition, a total of 400 small molecules were screened using the VapBC complex structure information. Furthermore, it was confirmed that the two final small molecule compounds effectively inhibited the formation of the VapBC complex at low concentrations.

Accordingly, the present invention provides a novel compound that inhibits the binding of VapB and VapC in the toxin-antitoxin complex VapBC of Klebsiella pneumoniae. Specifically, the compound can bind to the binding interface pocket of the antitoxin protein VapB located on the surface of the toxin protein VapC of Klebsiella pneumoniae and disrupt or inhibit the binding of the toxin-antitoxin complex VapBC.

The present inventors tried to prepare a novel antibacterial compound that destroys the VapBC complex by binding to a gap, not an active site of VapC, through the α2-α3 loop of VapB.

Accordingly, the antibacterial compound of the present invention may be the following compound 1 or 2.

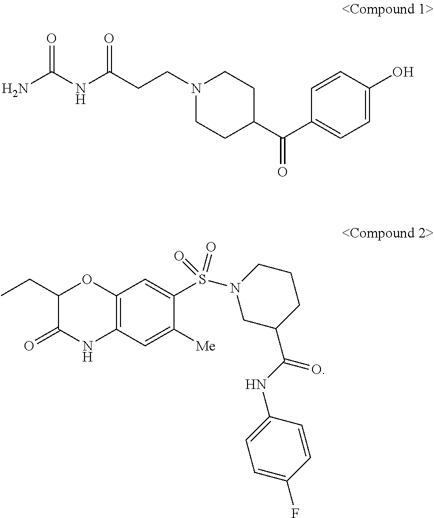 [SMILES: NC(=O)NC(=O)CCN1CCC(C(=O)c2ccc(O)cc2)CC1 ; CCC1Oc2cc(S(=O)(=O)N3CCCC(C(=O)Nc4ccc(F)cc4)C3)c(C)cc2NC1=O]

The peptide compound may exhibit antibacterial activity, particularly antibacterial activity against Klebsiella pneumoniae.

Therefore, the antibacterial peptide and antibacterial compound according to the present invention can be used for various purposes and uses requiring antibacterial activity, such as compositions for preventing, suppressing, improving or treating infectious diseases caused by bacteria, viruses, yeasts or fungi, animal feed compositions, animal feed additives, functional food compositions, food additives, food preservatives, disinfectants, sterilizing detergents, and the like, but not always limited thereto.

Accordingly, the present invention provides an antibacterial pharmaceutical composition comprising the antibacterial peptide or antibacterial compound. Preferably, the pharmaceutical composition may exhibit antibacterial activity against Klebsiella pneumoniae, and thus can be used for the prevention or treatment of pneumonia.

As used herein, the term “pneumonia” refers to inflammation of the lungs caused by infection with microorganisms such as bacteria, viruses, and fungi. Pneumonia is a disease that causes pulmonary symptoms that interfere with the normal function of the lungs, such as coughing, sputum caused by the discharge of inflammatory substances, and dyspnea due to breathing dysfunction, digestive symptoms such as nausea, vomiting, and diarrhea, and systemic disease throughout the body, such as headache, fatigue, muscle pain, and joint pain. The pneumonia that can be prevented or treated by the composition of the present invention may preferably be bacterial pneumonia, and more preferably, pneumonia caused by Klebsiella pneumoniae.

The term “Klebsiella pneumoniae” used in the present invention refers to a gram-negative short bacillus, and is a microorganism having a capsule without flagella and spores. This microorganism is a facultative anaerobe that usually grows well on agar, and is present in the human intestinal tract, oral cavity, etc., a causative bacterium of acute pneumonia, and is a microorganism isolated from bronchopneumonia, urinary tract infections, and the like.

The term “prevention” used in this invention refers to any activity that suppresses or delays the onset of the disease by the administration of the above composition, and “treatment” refers to any activity that improves or benefits the symptoms of the disease by the administration of the composition.

The pharmaceutical composition of the present invention can include a pharmaceutically acceptable carrier. The composition containing a pharmaceutically acceptable carrier may be in various oral or parenteral formulations. When formulated, it may be prepared using commonly used diluents or excipients such as fillers, extenders, binders, wetting agents, disintegrants, and surfactants. Solid formulations for oral administration are tablets, pills, powders, granules and capsules. These solid formulations are prepared by mixing one or more compounds with one or more suitable excipients such as starch, calcium carbonate, sucrose or lactose, gelatin, etc. Except for the simple excipients, lubricants, for example magnesium stearate, talc, etc, can be used. Liquid formulations for oral administrations are suspensions, solutions, emulsions and syrups, and the above-mentioned formulations can contain various excipients such as wetting agents, sweeteners, aromatics and preservatives in addition to generally used simple diluents such as water and liquid paraffin. Formulations for parenteral administration are sterilized aqueous solutions, water-insoluble excipients, suspensions, emulsions, lyophilized preparations and suppositories. Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerogelatin, etc.

In addition, the pharmaceutical composition of the present invention can have any one formulation form selected from the group consisting of tablets, pills, powders, granules, capsules, suspensions, solutions, emulsions, syrups, sterile aqueous solutions, water-insoluble excipients, suspensions, emulsions, lyophilized preparations, and suppositories.

The dosage of the pharmaceutical composition of the present invention is not particularly limited, and may vary depending on absorption in the body, body weight, patient's age, gender, health status, diet, administration time, administration method, excretion rate, severity of disease, etc. The pharmaceutical composition of the present invention is prepared in consideration of the effective amount range, and the formulated unit dosage preparation can be administered several times at regular intervals or using specialized medication methods according to the judgment of an expert who monitors or observes the administration of the drug as necessary and individual needs.

The present invention also provides an antibacterial quasi-drug comprising the peptide or compound as an active ingredient.

When the composition of the present invention is used as a quasi-drug additive, the peptide or compound may be added as it is or used together with other quasi-drugs or quasi-drug ingredients, and may be appropriately used according to the conventional method. The mixing ratio of active ingredients can be appropriately determined according to the purpose of use.

The quasi-drug composition of the present invention may preferably be a disinfectant cleaner, shower foam, gargreen, wet tissue, detergent soap, hand wash, humidifier filler, mask, ointment, patch, or filter filler, but not always limited thereto.

In addition, the present invention provides an antibacterial external preparation comprising the peptide or compound as an active ingredient.

The external preparation can further include fatty substances, organic solvents, solubilizers, thickeners and gelling agents, softening agent, antioxidants, suspending agents, stabilizers, foaming agents, fragrances, surfactants, water, ionic or non-ionic emulsifiers, fillers, metal ion blockers and chelating agents, preservatives, vitamins, blocking agents, wetting agents, essential oils, dyes, pigments, hydrophilic or lipophilic active agents, lipid vesicles, any other ingredients commonly used in external preparations, or conventionally used adjuvants.

The antibacterial pharmaceutical composition, quasi-drug, and antibiotic may exhibit antibacterial activity against Klebsiella pneumoniae.

Hereinafter, the present invention will be described in detail by the following examples. However, the following examples are only for illustrating the present invention, and the contents of the present invention are not limited thereto.

### Experimental Methods

The present invention can be carried out by the following experimental methods.

### 1. Cloning of VapB and VapC Genes, Protein Purification, and Mutant Generation

To confirm the structure of the VapBC complex, the genes encoding VapB (kpn_04185) and VapC (kpn_04186) of Klebsiella pneumoniae (MGH78578) were amplified by PCR using the primers VapB-F/VapB-R and VapC-F/VapC-R (Table 1).

TABLE 1
Primers used for cloning and mutant generation

| | | SEQ ID |
|---|---|---|
| Primer | Sequence | NO: |
| VapB-F^{a} | 5′-GGAATTCCATATGAGCATGATGGCTGAAAG-3′ | 3 |
| VapB-R^{1,b} | 5′-CCGCTCGAGTTACCACTCCTTGCGTAGC-3′ | 4 |
| VapB-R^{2,b} | 5′-CCGCTCGAGCCACTCCTTGCGTAGC-3′ | 5 |
| VapC-F^{a} | 5′-GGAATTCCATATGACCTCCGGATCTGCGC-3′ | 6 |
| VapC-R^{b} | 5′-CCGCTCGAGTTATTCAGGGTGGATTTCG-3′ | 7 |
| D9A-F^{a} | 5′-GGATCTGCGCTTTTTGATACCAATATTCTTATT-3′ | 8 |
| D9A-R^{b} | 5′-AATAAGAATATTGGTATCAAAAAGCGCAGATCC-3′ | 9 |
| E43A-F^{a} | 5′-CTGATTACCTGGATGGAGGTGATGGTTGGCGCT-3′ | 10 |
| E43A-R^{b} | 5′-AGCGCCAACCATCACCTCCATCCAGGTAATCAG-3′ | 11 |
| D90A-F^{a} | 5′-AAGCTTAAGCTGCCGGATGCCATCATTCTGGCG-3′ | 12 |
| D90A-R^{b} | 5′-CGCCAGAATGATGGCATCCGGCAGCTTAAGCTTT-3′ | 13 |
| D111A-F^{a} | 5′-ACGCGGAATACGAAGGATTTTGCCGGTATTCCT-3′ | 14 |
| D111A-R^{b} | 5′-AGGAATACCGGCAAAATCCTTCGTATTCCGCGT-3′ | 15 |

^{a,b}F and R represent the forward and reverse directions, respectively. The underline indicates the restriction enzyme site.

All PCR products and vectors were double-cleaved using the restriction enzymes Nde1 and Xho1. Then, the cleaved PCR products were connected to pET-21a (kpn_04185) and pET-28a (kpn_04186). The pET-28a vector used for VapC (kpn_04186) has an N-terminal tag (SEQ ID NO: 16, MGSSHHHHHHSSGLVPRGSH). The cloned plasmids were co-transformed into E. coli BL21 (DE3) cells (Novagen).

Bacterial cells were grown at 37° C. using Luria broth (LB) until the optical density at 600 nm (OD₆₀₀) reached 0.6. The cells were then induced by 0.5 mM IPTG and further cultured at 37° C. for 4 hours. The induced cells were centrifuged at 11,355 g and suspended in buffer A containing 500 mM NaCl with 20 mM Tris-HCl (pH 7.9) and 5% glycerol. The suspended cells were lysed by sonication and centrifuged at 28,306 g. After centrifugation, the supernatant containing soluble proteins was loaded in a Ni²⁺-affinity open column (Bio-Rad) previously equilibrated with buffer A. The bound protein was washed with buffer A containing 100 mM imidazole and eluted with an imidazole gradient (150-500 mM). The eluted protein was diluted with buffers containing 20 mM HEPES (pH 7.5) and 100 mM NaCl and further purified with a HiTrap Q HP anion exchange chromatography column (GE Healthcare) using a NaCl gradient (200-800 mM). The eluted protein was exchanged with a buffer containing 20 mM HEPES (pH 7.5) and 400 mM NaCl by size-exclusion chromatography on a HiLoad Superdex 200 prep-grade column (GE Healthcare) and concentrated to 20 mg/mL using an Amicon Ultra centrifugation filter unit (Millipore).

For the purification of the SeMet-labeled VapBC complexes, the same procedure as described above was used except that the cells were cultured in Nutrient Mix (Molecular Dimensions) and SeMet Medium Base containing excess SeMet.

Forward (F) and reverse (R) primers were used for D9A, E43A, D90A, and D111A to produce VapC mutants (Table 1). The mutants were produced using the EZchange Site-Directed Mutagenesis Kit (Enzynomics). The PCR product was ligated to pET-28a using an N-terminal tag (SEQ ID NO: 16, MGSSHHHHHHSSGLVPRGSH). VapC and mutated VapC proteins were prepared by the same steps as the proteins of the native VapBC complex. The purity of the purified protein was verified by sodium dodecyl sulfate-polyacrylamide gel electrophoresis at all steps.

### 2. Crystallization of VapBC Complex, Data Collection and Processing

To crystallize the VapBC complex, a sitting drop vapor diffusion method using Wizard Classic crystallization screen series (Rigaku) was used at 20° C. The final crystallization conditions were 0.1 M sodium citrate, pH 5.5 and 200 mM lithium sulfate. The crystals were cryoprotected with 20% glycerol and re-cooled in liquid nitrogen. Detailed information is provided in Tables 2 and 3 below.

### Data Collection Details

Diffraction data were collected using the ADSC Quantum Q270r CCD detector at beamlines 5C and 11C of Pohang Light Source (PLS), Korea. All raw data were adjusted and processed using HKL2000.

TABLE 2

| | | |
|---|---|---|
| Data set | SeMet | Native |

| | | |
|---|---|---|
| X-ray source | 5C beamline of | 5C beamline of |
| | PLS, Korea | PLS, Korea |

| | | |
|---|---|---|
| X-ray wavelength | 0.9794 | 0.9795 |

| | | |
|---|---|---|
| (Å) | | |
| Space group | C222₁ | C2 |

Unit cell parameters

| | | |
|---|---|---|
| a, b, c (Å) | 79.200, 110.323, | 110.435, 79.019, |
| | 59.703 | 59.609 |
| α, β, γ (°) | 90.000, 90.000, | 90.000, 89.985, |
| | 90.000 | 90.000 |
| Resolution range | 50.0-2.60 (2.64- | 50.0-2.00 (2.04- |
| (Å) | 2.60) | 2.00) |
| Molecules per | VapBC | 2 VapBC |
| ASU | heterodimer | heterodimers |

| | | |
|---|---|---|
| Observed | 110563 | 139747 |

reflections
(>1σ)

| | | |
|---|---|---|
| Unique | 8245 | 31667 |

reflections

| | | | | |
|---|---|---|---|---|
| <I/σ(I)> | 57.8 | (5.02)^{e} | 48.3 | (8.81)^{e} |
| Completeness (%) | 99.9 | (100.0)^{e} | 96.8 | (99.9)^{e} |
| Multiplicity^{a} | 13.4 | (10.8)^{e} | 4.4 | (4.7)^{e} |
| Rₚᵢₘ (%)^{b} | 5.4 | (63.2)^{e} | 3.7 | (26.7)^{e} |

| | | |
|---|---|---|
| CC_{1/2}, CC | (0.912, 0.977)^{e} | (0.890, 0.971)^{e} |

^{a}N_{obs}/N_{unique}
^{b}R_{merge} = Σ (I − <I>)/Σ<I>
^{e}The value in parentheses is the value of the shell with the highest resolution . . .

### Improved Statistical Results

During the structural calculation, the model was automatically constructed using PHENIX and the model was modified using Coot. REFMAC and PHENIX were used to obtain reasonable R_{work} and R_{free} values. MolProbity was used to verify the overall geometric structure. It was visualized using PyMOL (PyMOL Molecular Graphics System, Schroedinger, LLC., Cam-bridge, MA, USA).

TABLE 3

| Data set | SeMet | Native |
|---|---|---|
| R_{work}^{c} (%) | 26.8 | 24.4 |
| R_{free}^{d} (%) | 29.0 | 27.2 |
| No. of atoms/RMS | 1252/5.07 | 3192/2.72 |

RMSD^{f} from ideal geometry

| | | |
|---|---|---|
| Bond distance (Å) | 0.008 | 0.005 |
| Bond angle (°) | 1.099 | 1.056 |

Ramachandran statistics

| | | |
|---|---|---|
| Most favored regions (%) | 98.01 | 96.70 |
| Additional allowed regions | 1.99 | 3.30 |
| (%) | | |
| Residues in disallowed | 0.00 | 0.00 |
| regions (%) | | |
| PDB ID | 7BY2 | 7BY3 |

^{c}R_{work} = Σₕₖ₁||F_{obs}| − k|F_{calc}||/Σₕₖ₁|F_{obs}| (The degree of match between X-ray diffraction data and a model. The lower the value, the higher the match degree.)
^{d}R_{free} was calculated using the same method as R_{work} with 5% of data randomly extracted from the improvement results. (The degree of match between randomly selected data and a model.)
^{f}Root-mean-square deviation (RMSD) was calculated using REFMAC. (The degree to which the structure of the determined model matches the bond lengths and angles of typical molecules.)

3. Growth Analysis and Colony Forming Unit (cfu) Measurement

The gene encoding VapC was additionally cloned into pBAD33. The cloned plasmid was transformed into competent E. coli BL21 (DE3) cells containing the previously cloned gene encoding VapB. This compatible plasmid pair (pBAD33 and pET-21a) was incubated overnight at 37° C. in M9 minimal medium containing chloramphenicol (25 μg/mL) and ampicillin (50 μg/mL). The culture product grown overnight was diluted 50-fold and further incubated for another 5 hours. When OD₆₀₀ was 0.2, 0.2% arabinose was added. For growth analysis, 0.5 mM IPTG was added after 1 hour. For cfu measurement, cell samples were collected every 30 minutes and streaked onto LB medium plates containing 0.2% glucose (arabinose) and 0.5 mM IPTG. The plates were counted after 16 hours of incubation.

4. Confirmation of Klebsiella pneumoniae VapBC Complex Structure

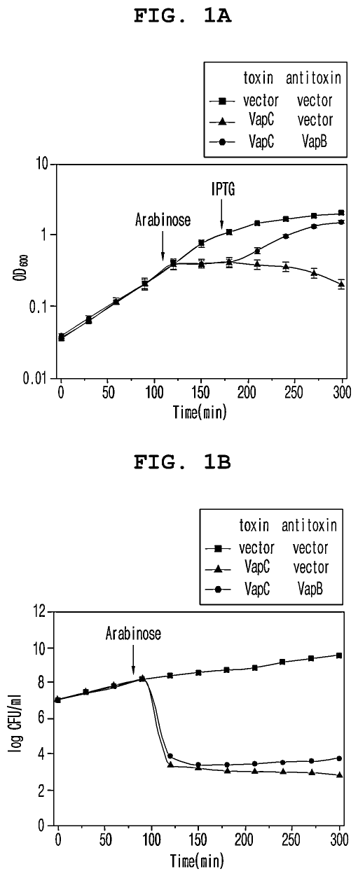

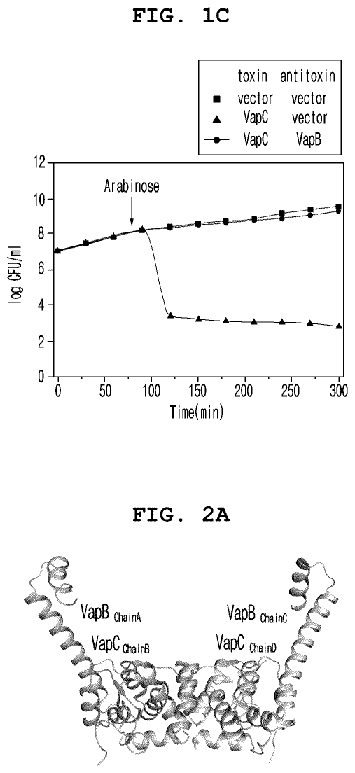

The Klebsiella pneumoniae VapBC TA system was experimentally verified. The expression of VapC significantly inhibited cell growth and reduced colony forming units (cfu). However, the suppressed cell viability was almost completely restored by the expression of VapB (FIG. 1). The bacteria used for verification of the Klebsiella pneumoniae VapBC TA system was E. coli.

To interpret the structure of VapBC, single wavelength anomalous diffraction (SAD) data for the selenomethionine (SeMet)-labeled VapBC complex were refined [Protein Data Bank (PDB) ID: 7BY2] and used to solve the phase problem. Then, native data were refined through molecular replacement using the structure of the SeMet-labeled VapBC complex as a search model (PDB ID: 7BY3).

As a result of structural calculation of Klebsiella pneumoniae VapBC from native data, the overall form of the VapBC complex was obtained. The VapB antitoxin is divided into an N-terminal region containing a DNA binding domain and a C-terminal region containing a toxin binding site blocking the active site of the VapC toxin. Interestingly, the phase structure of VapBC in the SAD data showed an incomplete complex structure in which the C-terminus of VapB was decomposed, so the active site of VapC was not inhibited by VapB.

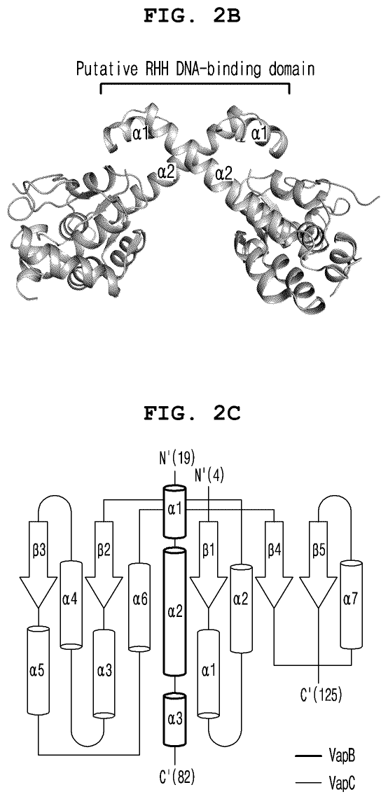

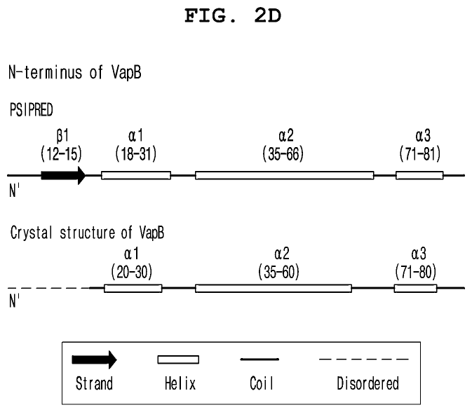

In the above crystal structure, two types of intermolecular interfaces originating from crystal packing were identified. The first one is a toxin dimerization-driven heterotetramer (FIG. 2A). In this case, the interaction between α-helices in adjacent toxin monomers is the main binding force. The other one is an antitoxin dimerization-driven heterotetramer mediated by the DNA binding domain of the antitoxin (FIG. 2B).

A schematic diagram of the VapBC heterodimer helps to understand the spatial arrangement of the secondary structural elements that make up the heterodimeric architecture (FIG. 2C). In the native structure, the first β-strand of VapB N-terminus was not observed in the map because of its low electron density. According to the prediction using PSIPRED, VapB is expected to have RHH DNA-binding domains in the order of β1-α1-α2 at the N-terminus (FIG. 2D).

### 5. In Vitro RNase Analysis for VapC and VapC Mutants

The RNase activity of VapC and VapC mutants was confirmed using an RNase Alert Kit (Integrated DNA Technologies, Inc.). In this analysis, each well contained 50 pm of synthetic RNA with a fluorophore at one end and a quencher group at the other end. The quencher was separated when the prepared RNA was contacted by RNase. Then, fluorescence (RFU) generated during the 1 hour reaction period was detected using a SPECTRAmax GEMINI XS spectrometer (Molecular Devices) at 490 nm (excitation wavelength) and 520 nm (emission wavelength), respectively. For this analysis, the purified VapC and VapC mutants were treated with 10 mM EDTA to remove metal ions and exchanged with a purification buffer containing 10 mM Mg²⁺. In each experiment, the concentrations of both VapC and VapC mutants were 10 μM. To prevent contamination, 40 units of RiboLock (Thermoscientific) were added to each well. RiboLock does not inhibit prokaryotic RNase I and H. VapC is not affected by RiboLock because it is structurally similar to RNase H. The final concentration of RNA was 5 pM, and the buffer components were 20 mM HEPES (pH 7.5) and 400 mM NaCl.

6. Structural Analysis by Comparison of Klebsiella pneumoniae VapBC Complex and its Homologues

In order to confirm the structural characteristics of Klebsiella pneumoniae VapBC, a comparative analysis with its structural homologues was performed and the structure of the Klebsiella pneumoniae VapBC complex was confirmed. In order to reflect the latest information, it was compared with relatively recently reported homologues since 2017. First, amino acid sequence alignment of VapC toxin was performed using Clustal Omega 1.2.1 and ESPript 3.0. In this alignment, the primary sequences of M. tuberculosis VapC11 (PDB ID: 6A7V) and VapC26 (PDB ID: 5X3T), C. crescentus VapC1 (PDB ID: 5K8J), and Hemophilus influenza VapC1 (PDB ID: 6NKL) were compared to the sequence of Klebsiella pneumoniae VapC.

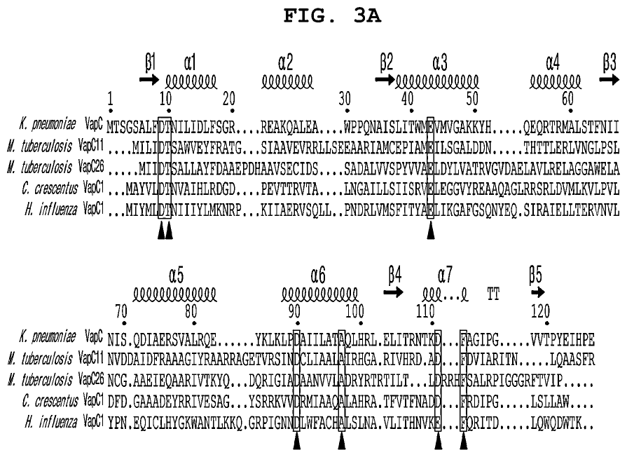

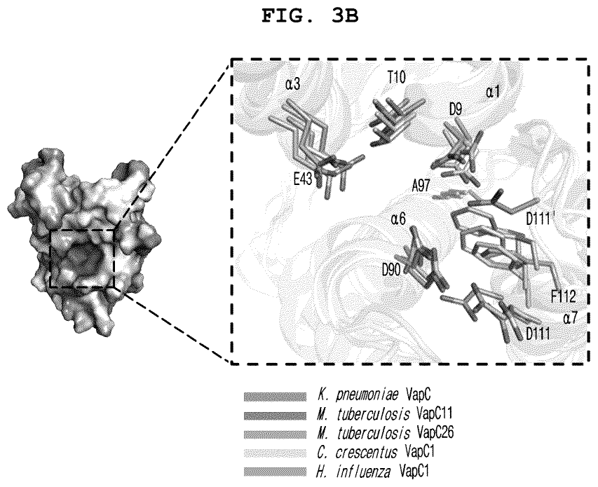

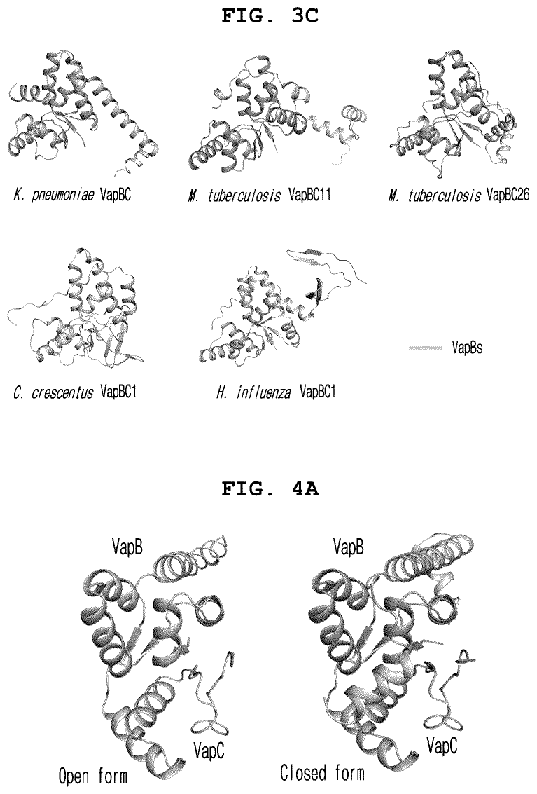

FIG. 3 shows the structural comparison of the Klebsiella pneumoniae VapBC and its homologues. FIG. 3A shows the sequence alignment of Klebsiella pneumoniae VapC and its homologues, revealing seven highly conserved residues (marked with triangles). The first, third, fourth, and sixth conserved residues are negatively charged and acidic residues such as aspartate and glutamate constitute the active site of the toxin. Among the remaining three residues, the positions of the second threonine and the seventh phenylalanine are closely related to the position of the active site. Hydrogen bonding and hydrophobic interactions between these residues and active site residues serve as the basis for the construction and structural integrity of the active site and affect them (FIG. 3B).

Despite the low sequence similarity (average of 13.7%) with the four homologues, Klebsiella pneumoniae VapC shares structural similarity with other VapC proteins. The VapC toxin has a standard α/β/α sandwich fold consisting of 5 parallel β-strands composed of 7 α-helices. The VapC toxin also features a PIN domain essential for ribonuclease activity. The conserved acidic residues of the PIN domain proteins coordinate with divalent metals and induce catalytic activity based on the acid-base reaction.

The four homologues were also compared to Klebsiella pneumoniae VapBC to confirm the differences in VapB structure and VapB and VapC binding patterns (FIG. 3C). As a result of the comparison, the VapC structure had similar folding, but the binding pattern between the VapC toxin and its VapB and VapB DNA binding domains was quite different. The C-terminal toxin binding region of VapB is inherently very flexible, so structural folding and integrity are achieved only after binding to the toxin. For the above reasons, the α-helix contents and loop contents of each VapB in each binding region between these VapB antitoxin and VapC toxin appear to have a large difference. VapB also has two other DNA binding domains including RHH and AbrB type domains. Interestingly, the DNA binding domain of VapB varies not only by the domain type but also within the same domain type, and the orientation of the domain is significantly different depending on the curve of the hinge region. This emphasizes that the structural variability of the VapB protein causes diversity in transcriptional regulatory mechanisms and further explains the specificity of its own promoter region recognition.

### 7. Confirmation of RNase Activity of VapC and Inhibitory Mechanism of VapB

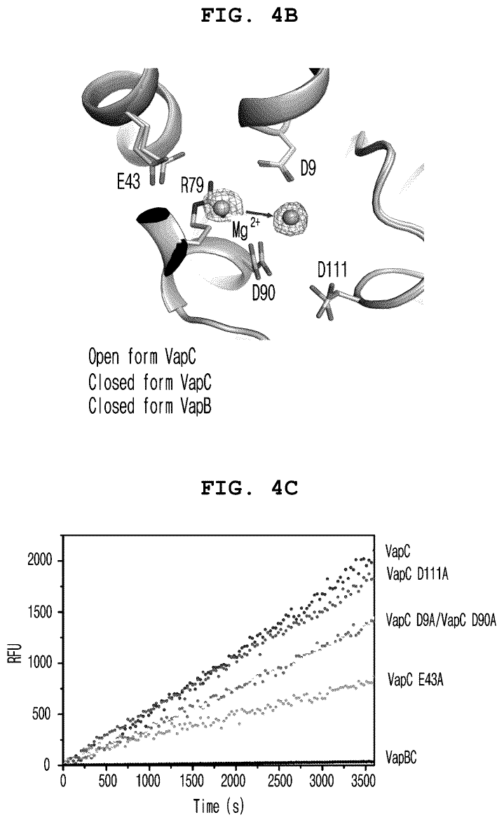

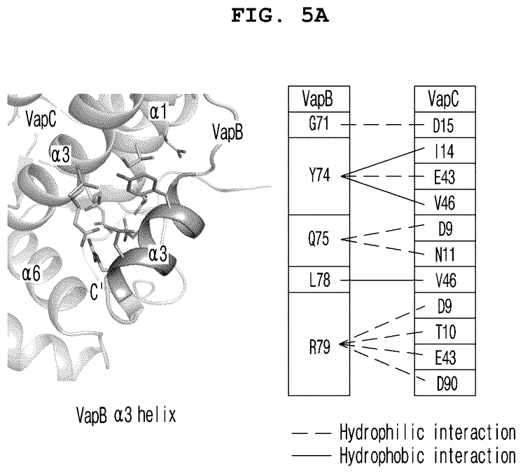

The structure obtained from the SAD data is an open type with the active site exposed because the C-terminus of VapB is naturally decomposed (FIG. 4A). On the other hand, the native structure obtained from the native crystal is a closed type with the active site in which the toxin-binding region of VapB is completely conserved (FIG. 4A). Depending on the innate nature of VapC, RNase activity appears only in the presence of a divalent metal such as Mg²⁺.

Klebsiella pneumoniae VapC showed RNase activity only when Mg²⁺ was added to the protein solution. In the open type without VapB interference at the active site, Mg²⁺ ions are located in the middle of the conserved active site residues (D9, E43 and D90) of VapC. However, in the closed type, VapB covers the active site of the toxin. R79 of VapB precisely blocks the original Mg²⁺ site and transfers Mg²⁺ to the center of the residues D9, D90 and D111 of VapC, allowing the active site to switch from an open state to a closed state (FIG. 4B). This inhibition mechanism of the active site is a new TA system mechanism. This inhibition mechanism of the active site is observed for the first time through the crystal structure of two states.

To expand the significance of these results, each residue containing the active site of VapC was mutated, and the RNase activity of each VapC mutant was measured (FIG. 4C). In the graph showing the kinetics over 1 hour and showing the proportional chart for VapC, the residue that contributed the most to the activity was E43 and the residue that contributed the least was D111. In conclusion, VapC active site inhibition is achieved by completely eliminating the contribution of E43 to RNase activity, since R79 of VapB causes the substitution of Mg²⁺. However, mutation experiments showed that VapC still had about 40% of the RNase activity when E43 of VapC was mutated to alanine. This suggests that the RNase activity of VapC depends on the location of Mg²⁺ that can be replaced by VapB as well as the contribution of each residue constituting the active site.

### EXAMPLE 1

### Design of VapBC Binding Interface Mimetic Peptide

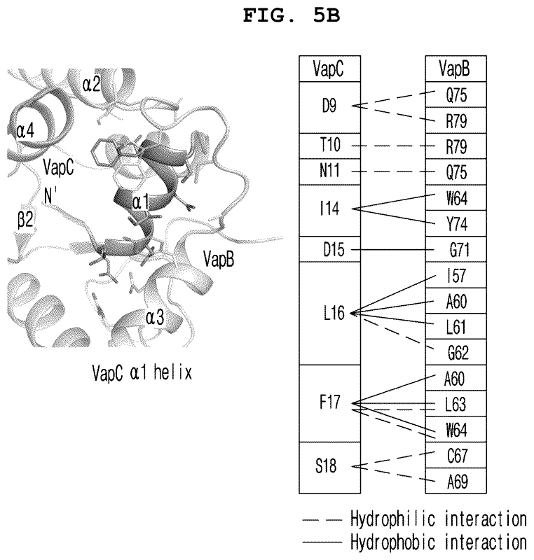

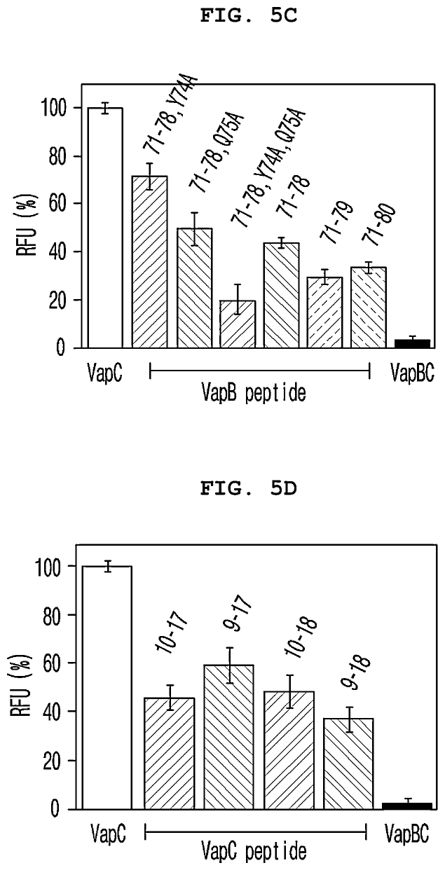

If the formation of the TA complex is artificially inhibited, the toxin released freely from the TA complex may inhibit growth or eventually lead to bacterial death. Therefore, in order to design peptides capable of disrupting the Klebsiella pneumoniae VapBC complex, VapB and VapC were analyzed based on the analysis results of proteins, interfaces, structures and assemblies (PISA) and the protein interaction calculator (PIC) server. The results showed that the α3 helix of VapB (FIG. 5A) and the al helix of VapC (FIG. 5B) were the most important in the dimerization interaction between VapB and VapC.

Therefore, several peptides were designed based on the order of the α3 helix of VapB and the α1 helix of VapC (Table 4).

TABLE 4

| | | | | SEQ |
|---|---|---|---|---|
| | Residues (N- to C- | Mimicked | Mimicked | ID |
| Name | terminus) | protein | region | NO: |
| VapB⁷¹⁻⁷⁸ | GMEYQRQL (71-78) | VapB | α3 | 17 |
| VapB^{71-78, Y74A} | GMEAQRQL (71-78) | VapB | α3 | 18 |
| VapB^{71-78, Q75A} | GMEYARQL (71-78) | VapB | α3 | 19 |
| VapB^{71-78, Y74A, Q75A} | GMEAARQL (71-78) | VapB | α3 | 20 |
| VapB⁷¹⁻⁷⁹ | GMEYQRQLR (71-79) | VapB | α3 | 21 |
| VapB⁷¹⁻⁸⁰ | GMEYQRQLRK (71-80) | VapB | α3 | 22 |
| VapC¹⁰⁻¹⁷ | TNILIDLF (10-17) | VapC | α1 | 23 |
| VapC⁹⁻¹⁷ | DTNILIDLF (9-17) | VapC | α1 | 24 |
| VapC¹⁰⁻¹⁸ | TNILIDLFS (10-18) | VapC | α1 | 25 |
| VapC⁹⁻¹⁸ | DTNILIDLFS (9-18) | VapC | α1 | 26 |

The peptides were designed through structural analysis and synthesized by ANYGEN (http://www.anygen.com).

### EXAMPLE 2

### Analysis of VapBC Complex Disruption by VapBC Binding Interface Mimetic Peptides

In this example, the peptide (100 μM) synthesized in Example 1 was added to the VapBC complex (10 μM) and the RFU of the activated VapC toxin was measured. Other experimental protocols were the same as those for the RNase activity test of VapC and VapC mutants (Experimental Method 5). Upon addition of the peptide of the present invention to the VapBC complex, the inhibitory efficacy of the peptide can be estimated by measuring the RNase activity generated by the free VapC released by the disruption of the VapBC complex.

### 2.1 Analysis of VapBC Complex Disruption Efficacy of VapB α3 Helix-Based Mimetic Peptides

In the VapBC complex disruption assay, the α3 helix of VapB consisted of G71-K80 (GMEYQRQLRK, SEQ ID NO: 22), but the VapB⁷¹⁻⁷⁸ peptide consisted of G71-L78 (GMEYQRQL, SEQ ID NO: 17), which excluded R79 of VapB interacting with three active site residues (D9, E43 and D90) of VapC. Y74 and Q75 of VapB interacted with E43 and D9 of VapC, respectively.

As shown in FIG. 5C, all of the VapB mimetic peptides represented by SEQ ID NO: 17 to NO: 22 showed an effect of increasing free VapC toxins by destroying the VapBC complex. Among them, when Y74 and Q75 were individually mutated to alanine, the VapB^{71-78, Y74A} peptide (SEQ ID NO: 18) showed the highest efficacy among all the designed peptides. The VapB^{71-78, Y74A, Q75A} peptide (SEQ ID NO: 20) in which both Y74 and Q75 were mutated to alanine showed lower efficacy than the VapB peptide in which either one was mutated to alanine, which may be due to a defect in the structural integrity of the α-helix because of multiple alanine substitutions.

### 2.2 Efficacy Assay of VapC α1 Helix-Based Mimetic Peptides

In the VapBC complex structure, the al helix of VapC is composed of T10-F17 (TNILIDLF, SEQ ID NO: 23). Although D9 and S18 were not included in the al helix of VapC, the peptides represented by SEQ ID NO: 24 to NO: 26 including D9 and S18 interacting with VapB were designed.

As shown in FIG. 5D, all of the VapC mimetic peptides represented by SEQ ID NO: 17 to NO: 23 showed an effect of increasing free VapC toxins by destroying the VapBC complex.

### EXAMPLE 3

### Analysis of Effect on Free VapC Activity

Since the peptides of the present invention are made by mimicking the binding interface of VapB and VapC, arithmetically, the more residues that participate in the binding of VapB and VapC are present in the peptide, the greater the ability to disrupt the complex. However, if the peptide contains the residues that interact with the nucleic acid binding site of VapC, which is the active site of VapC, the destructive ability is suppressed. Accordingly, in this example, the effect of the peptide of the present invention on the activity of free VapC was analyzed.

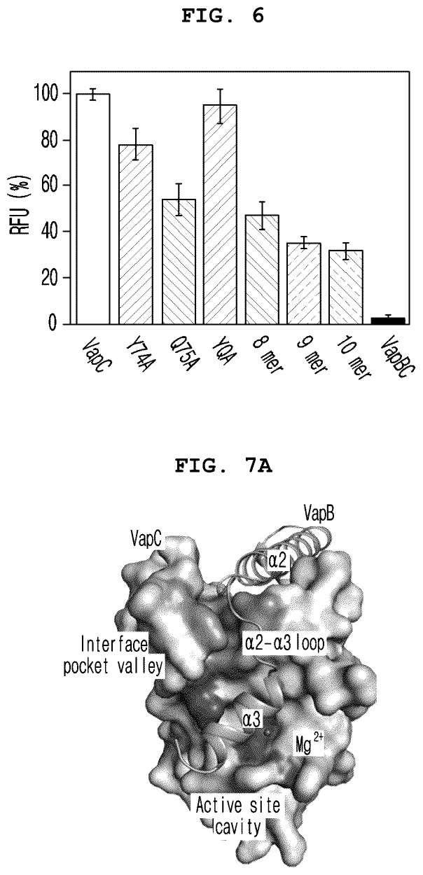

When the free VapC was treated with a VapB-mimetic peptide, the overall RNase activity measured was slightly increased, but the pattern was similar to the results according to Example 2.1 (FIG. 6). In particular, defects in the structural integrity due to multiple alanine substitutions (both Y74A and Q75A) had little effect on the activity of VapC.

From the above results, it was confirmed that the VapB-mimetic peptides represented by SEQ ID NO: 17 to NO: 22 showed the effect of controlling Klebsiella pneumonia.

### EXAMPLE 4

### Structure-Based Pharmacophore Generation and Molecular Docking Studies

Structure-based drug design was performed with structure-based pharmacophore (SBP) models and molecular docking simulations to virtually screen key hit compounds. SBP modeling, which extracts chemical features from the protein-ligand complex structure, was performed to generate an SBP model for the crystal structure of the VapBC complex (PDB ID: 7BY3).

The receptor-ligand pharmacophore generation was performed using the pharmacophore generation tool of Discovery Studio (DS) 2018 software (DASSAULT SYSTEMS, 2017) with default parameters. An SBP model with four important chemical features [2 hydrogen bond (HB) donors, 1 HB receptor, and 1 hydrophobic interaction] was designed by considering only the VapB α2-α3 loop region (W64, C67 and Y74 of VapB) as an alternative ligand that inhibits protein-protein interactions. Using the generated SBP model, a total of ˜610,000 compounds were searched and ˜16,000 compounds were selected through filtering.

Then, molecular docking simulations were performed with 7155 different sets of compounds representing a total of ˜610,000 compounds and the generated compounds. The target binding site of VapC was established in the cocrystal structure of VapB, especially in the α2-α3 loop region with a radius of 14 Å. The 3D structure of VapC was improved and protonated by the Prepare Protein tool implemented in DS 2018. CDOCKER was used in DS 2018 to perform docking simulations. CDOCKER is a CHARMM-based docking tool that uses a robust ligand flexibility algorithm and an improvement process that performs arbitrary rotation and grid-based simulation annealing at high temperatures. The resulting hit compounds were ranked by score, which takes into account both the fit value and the negative CDOCKER interaction energy (including van der Waals and electrostatic interactions).

### EXAMPLE 5

### Analysis of VapBC Complex Disruption of Small Molecule Compounds Closing Interface Pocket and Selection of Compounds

To disrupt the VapBC complex and activate the VapC toxin, 400 small molecules were initially selected by virtual screening using a pharmacophore model and molecular docking. In this virtual screening, the small molecules predicted to close the interface pocket of the VapBC complex were explored, and the small molecules were listed in order of virtual fitness scores as a criterion for selecting initial candidates.

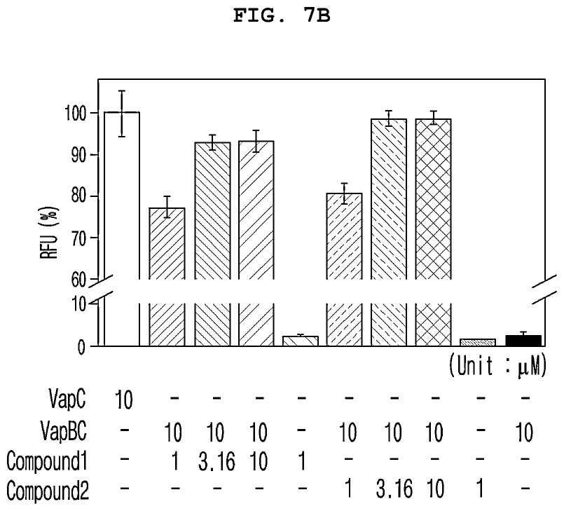

Most of these compounds had an average of ˜5.1 HB receptors, ˜2.2 HB donors, and 6.8 rotatable bonds, and were distributed in the MW range of 300-500 Da. The predicted A log P values for most of the compounds were in the range of 0.5-5, and the mean value was 2.8. Each compound was designed to destroy the VapBC complex by binding to the gap through the α2-α3 loop of VapB rather than the active site cavity of VapC to connect the interface pocket (FIG. 7A).

Using a similar analysis method as in the design of peptides that activate VapC, the inhibitory effect of the compounds was ranked according to the degree of RNase activity by the free VapC released due to the disruption of the VapBC complex. That is, the small molecules were added to the VapBC complex (10 μM) and the efficacy of the small molecules to activate VapC was tested. Other experimental protocols were the same as those described for the peptide-induced complex disruption assay (Examples 2 and 3).

Through three tests, a total of 400 compounds were narrowed down to 25 according to the inhibitory efficacy ranking. These 25 small molecules were further narrowed down through three additional tests to the two compounds with the highest effect, named compound 1 and compound 2 (FIG. 7B). These two final compounds did not affect the RNase activity of VapC when added to free VapC because they were designed to bind to the binding interface between VapB and other regions of VapC, rather than the active site of VapC (FIG. 8).

The two compounds finally selected through this are as follows.

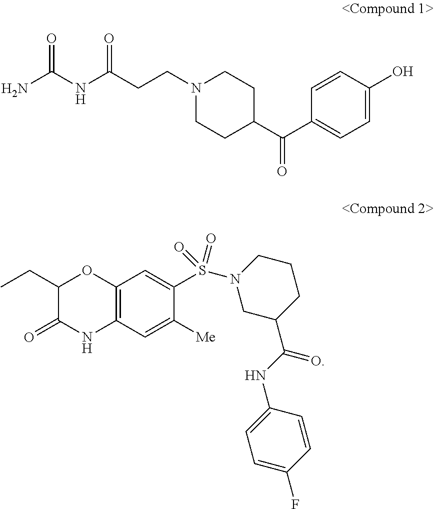 [SMILES: NC(=O)NC(=O)CCN1CCC(C(=O)c2ccc(O)cc2)CC1 ; CCC1Oc2cc(S(=O)(=O)N3CCCC(C(=O)Nc4ccc(F)cc4)C3)c(C)cc2NC1=O]

### EXAMPLE 6

### Synthesis of Compounds 1 and 2

### General Matters

Silica gel column chromatography was performed on silica gel 60, 230-400 mesh, Merck. ¹H and ¹³C NMR spectra were recorded on JEOL 400 MHz. Chemical shifts were reported in ppm using Me₄Si as a reference standard. Mass spectra were recorded on a 6460 triple quad LC-MS instrument.

### Synthetic Formula 1. Synthesis of Compound 1 (N-carbamoyl-3-(4-(4-hydroxybenzoyl)piperidine-1-yl)propanamide)

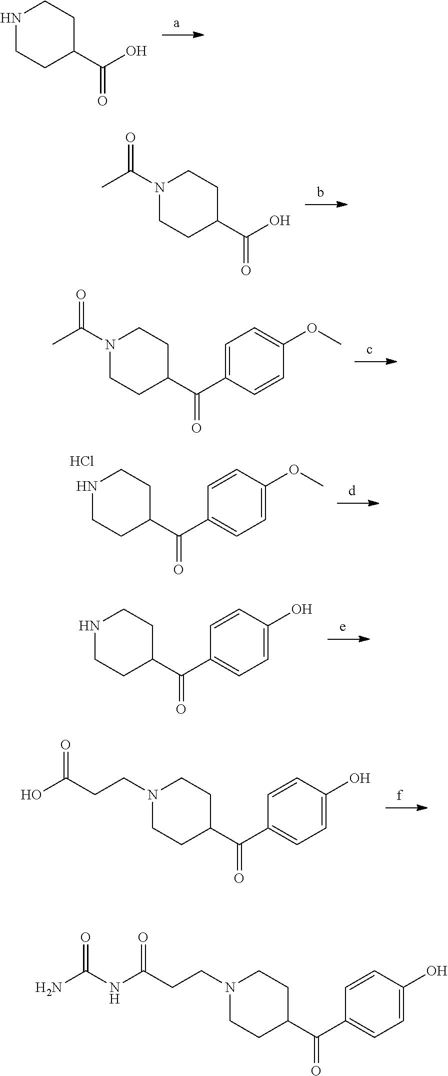

Reagents and conditions: (a) Ac₂O, reflux, 7 h, 85%; (b) (i) SOCl₂, 1,2-dichloroethane 65° C., 1 h; (ii) anisole, AlCl₂, reflux, 10 h, 44%; (c) 1M HCl, reflux, 13 h, 96%; (d) 48% HBr, AcOH, reflux, 48 h, 73%; (e) methyl acrylate, CH₂Cl₂, rt, overnight, 36%; (f) urea, Ac₂O, rt, 18 h, 90%.

### 1-Acetyl-4-piperidine carboxylic acid

A solution of 4-piperidine carboxylic acid (38.75 g, 0.3 mol, 1 eq) in anhydrous acetic acid (Ac₂O) (150 mL, 1.36 mol, 4.53 eq) was refluxed for 7 hours and stirred overnight at ambient temperature. The mixture was then concentrated using a rotary evaporator, triturated in diethyl ether (Et₂O), and recrystallized from diisopropyl ether (DIPE) and methanol (MeOH). A product (85%) was obtained by filtration with a glass filter.

¹H NMR (400 MHz, CDCl₂): δ 9.89 (s, 1H), 4.48-4.32 (m, 1H), 3.80 (d, J=7.7 Hz, 1H), 3.26-3.08 (m, 1H), 2.95-2.78 (m, 1H), 2.64-2.47 (m, 1H), 2.11 (s, 3H), 1.98 (d, J=7.7 Hz, 2H), 1.82-1.57 (m, 2H);

¹³C NMR (101 MHz, CDCl₃): δ 178.10, 169.47, 60.71, 45.67, 40.97, 40.55, 28.34, 27.77, 21.27.

### N-acetyl-4-(4-methoxybenzoyl)piperidine

A solution of SOCl₂ (2.97 g, 25 mmol, 1 eq) in 1,2-dichloroethane (10 mL) was added dropwise to a solution of 1-acetyl-4-piperidine carboxylic acid (4.28 g, 25 mmol, 1 eq) and preheated to 40° C. The temperature of this mixture was increased to 65° C., stirred for 1 hour, and air-cooled to ambient temperature. The resulting acid chloride was reacted with anisole (2.70 g, 25 mmol, 1 eq). Anhydrous aluminum trichloride (AlCl₃) (6.66 g, 50 mmol, 2 eq) was slowly added thereto, the reaction mixture was refluxed for 10 hours, stirred overnight at ambient temperature, and the mixture was poured into ice water. The mixture was extracted with dichloromethane (DCM) and brine, dried over MgSO₄, and rotary-evaporated to obtain a product (44%).

¹H NMR (400 MHz, CDCl₃): δ 7.94 (d, J=8.9 Hz, 2H), 7.05 (d, J=8.9 Hz, 2H), 3.86 (s, 3H), 3.48 (m, 4H), 3.05 (m, 1H), 2.13 (s, 3H), 1.98-1.58 (m, 4H).

¹³C NMR (101 MHz, CDCl₃): δ 200.16, 168.93, 163.62, 130.57, 128.59, 113.96, 55.53, 45.86, 42.84, 41.09, 28.76, 28.55, 21.51.

### 4-(4-Methoxybenzoyl)piperidinium chloride:

N-acetyl-4-(4-methoxybenzoyl)piperidine (2.09 g, 8 mmol, 1 eq) was dissolved in 1 M aq. HCl (90 mmol, 90 eq), refluxed for 13 hours and stirred overnight at ambient temperature. The reaction mixture was concentrated by rotary evaporation using ethanol (EtOH) to give the desired amine in the form of HCl salt (96%).

¹H NMR (400 MHz, MeOD): δ 8.05 (d, J=8.8 Hz, 2H), 7.07 (d, J=8.8 Hz, 2H), 3.91 (s, 3H), 3.79 (t, J=10.9 Hz, 1H), 3.49 (d, J=12.8 Hz, 2H), 3.23 (t, J=12.8 Hz, 2H), 2.11 (d, J=12.8 Hz, 2H), 1.93 (q, J=12.8 Hz, 2H).

### 4-(4-Hydroxy)benzoylpiperidine

A solution of 4-(4-methoxy)benzoylpiperidine hydrochloride (3.0 g, 11.7 mmol) in acetic acid (16 mL) and 48% HBr (aq) (16 mL) was reflux-heated for 48 hours. The reaction mixture was evaporated and dried in vacuo to give a gray-white solid suspended in saturated NaHCO₃ (aq). The resulting precipitate was collected by filtration, washed with water, and dried to give a product (73%).

¹H NMR (400 MHz, CDCl₃): δ 9.68 (s, 1H), 7.77 (d, J=8.8 Hz, 2H), 6.78 (d, J=8.8 Hz, 2H), 3.00 (m, 1H), 1.69-1.79 (m, 4H), 1.77-1.52 (m, 4H).

Mass spectrum: Found 206 [M+H]⁺, C12H15NO2 requires 205

### 3-(4-(4-Hydroxybenzoyl)piperidine-1-yl)propionic acid

4-(4-Hydroxy)benzoylpiperidine (2.0 g, 9.7 mmol) and methyl acrylate (1.0 g, 19.4 mmol, 1.2 eq) were added to DCM (15 ml). The mixture was stirred overnight at room temperature. After evaporation under reduced pressure, the corresponding methyl ester was obtained and hydrolyzed by heating at 40° C. for 30 minutes with 5% NaOH solution. At the end of the reaction, the reaction mixture was cooled to room temperature and neutralized with HCl. The resulting precipitate was filtered, dried and used without further purification (36%).

¹H NMR (400 MHz, CDCl₃): δ 12.27 (br, 1H), 9.68 (s, 1H), 7.77 (d, J=8.8 Hz, 2H), 6.78 (d, J=8.8 Hz, 2H), 3.64 (m, 2H), 3.00 (m, 1H), 2.47-2.24 (m, 6H), 1.77-1.52 (m, 4H).

Mass spectrum: Found 278 [M+H]⁺, C15H19NO4 requires 277

### N-carbamoyl-3-(4-(4-hydroxybenzoyl)piperidine-1-yl)propanamide

3-(4-(4-Hydroxybenzoyl)piperidine-1-yl)propionic acid (2.0 g, 7.2 mmol) was treated with urea (0.4 g, 7.9 mmol, 1.1 eq) in 5 mL of acetic anhydride. The reaction mixture was heated to 90° C. for 30 minutes. Five minutes later, a white precipitate was formed. Then, 8 mL of water was added thereto and stirred continuously for an additional 5 minutes. After cooling the solution to room temperature, the precipitate was filtered and dried under high vacuum overnight to give a product (90%).

¹H NMR (400 MHz, CDCl3): δ 10.38 (s, 1H), 9.68 (s, 1H), 7.77 (d, J=8.8 Hz, 2H), 7.35 (s, 2H), 6.78 (d, J=8.8 Hz, 2H), 3.64 (m, 2H), 3.00 (m, 1H), 2.47-2.24 (m, 6H), 1.77-1.52 (m, 4H).

Mass spectrum: Found 320 [M+H]⁺, C16H21N3O4 requires 319

### Synthetic Formula 2. Synthesis of Compound 2 (1-((2-ethyl-6-methyl-3-oxo-3,4-dihydro-2H-benzo[b][1,4]oxazine-7-yl)sulfonyl)-N-(4- fluorophenyl)piperidine-3-carboxamide)

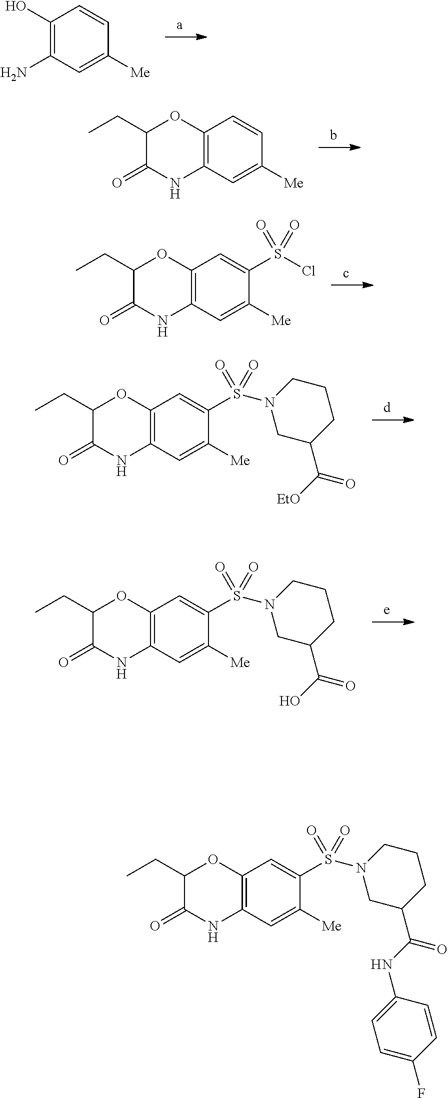

Reagents and conditions: (a) Ethyl 2-bromobutyrate, DBU, NMP, microwave, 180° C., 5 min, 76%; (b) HSO₃Cl, 0° C., 1 h, 66%; (c) ethyl piperidine-3-carboxylate, TEA, MC, rt, 2 h, 94%; (d) LiOH, THF, rt, ON, 92%; (e) BOP Reagent, 4-fluoroanilne, DMIPA, THF, rt, 18 h, 20%.

### 2-Ethyl-6-methyl-2H-benzo[b][1,4]oxazine-3(4H)-one

A mixture of ethyl 2-bromo butyrate (97.5 mg, 0.50 mmol), 2-amino-4-methylphenol (73.9 mg, 0.60 mmol), and 1,8-diazabicyclo[5.4.0]undec-7-ene (DBU, 83.7 mg, 0.55 mmol) in distilled NMP (2 mL) was filled into a 10 mL process vial and sealed with a cap with a septum. The loaded vial was placed in a cavity of the microwave reactor and heated at 180° C. for 3 minutes in a fixed mode. The reaction mixture was diluted with EtOAc (10 mL) and washed with brine (3×5 mL). The combined organic layer was dried over MgSO₄ and condensed under reduced pressure. The residue was purified by flash column chromatography on a silica gel using EtOAc-hexane as an eluent to give a product (76%).

¹H NMR (400 MHz, CDCl₃): δ 9.87 (br s, 1H), 7.00-6.85 (m, 3H), 4.68 (q, J=6.8 Hz, 1H), 2.27 (s, 3H), 1.59 (m, 2H), 0.89 (m, 3H).

Mass spectrum: Found 192 [M+H]⁺, C11H13NO2 requires 191

### 2-Ethyl-6-methyl-3-oxo-3,4-dihydro-2H-benzo[b][1,4]oxazine-7-sulfonyl chloride

2-Ethyl-6-methyl-2H-benzo[b][1,4]oxazine-3(4H)-one (2.5 g, 13.4 mmol) was added to sulforochloride acid (10 mL) in several batches for 20 minutes at 0° C., and the dye reaction mixture was maintained for 1 hour. The reaction mixture was carefully poured onto ice (100 g) and the resulting mixture was extracted with dichloromethane (100 mL). The organic layer was dried (sodium sulfate) and concentrated to give a product (66%).

¹H NMR (400 MHz, CDCl₃) δ 9.87 (s, 1H), 7.71 (s, 1H), 7.52 (s, 1H), 4.68 (q, J=6.8 Hz, 1H), 2.27 (s, 3H), 1.59 (m, 2H), 0.89 (m, 3H).

Mass spectrum: Found 290 [M+H]⁺, C11H12C1NO4S requires 289

### Ethyl 1-((2-ethyl-6-methyl-3-oxo-3,4-dihydro-2H-benzo[b][1,4]oxazine-7-yl)sulfonyl)piperidine-3-carboxylate

2-Ethyl-6-methyl-3-oxo-3,4-dihydro-2H-benzo[b][1,4]oxazine-7-sulfonyl chloride (1.83 g, 6.36 mmol), ethyl piperidine-3-carboxylate (1.00 g, 6.36 mmol) and triethylamine (2.66 mL, 19.1 mmol) in methylene chloride (10.0 mL) were stirred at room temperature for 2 hours. The mixture was quenched with water and then extracted with ethyl acetate. The extract was washed sequentially with 1 N HCl solution, water, saturated sodium bicarbonate solution, water and brine. The extract was then dried over sodium sulfate (anhydrous). After filtration, the filtrate was concentrated to give a product (94%).

¹H NMR (400 MHz, CDCl₃) δ 9.87 (s, 1H), 7.71 (s, 1H), 7.52 (s, 1H), 4.70 (q, J=6.8 Hz, 1H), 4.21 (m, 2H), 3.50 (m, 2H), 3.25 (m, 2H), 2.64 (s, 3H), 2.33 (m, 1H), 2.09-1.98 (m, 4H), 1.53 (m, 2H), 1.21 (m, 3H), 0.89 (m, 3H).

Mass spectrum: Found 411 [M+H]⁺, C19H26N2O6S requires 410

### 1-((2-Ethyl-6-methyl-3-oxo-3,4-dihydro-2H-benzo[b][1,4]oxazine-7-yl)sulfonyl)piperidine-3-carboxylic acid

A 1 M aqueous solution of lithium hydroxide (3.5 mL) was added to a solution of ethyl 1-((2-ethyl-6-methyl-3-oxo-3,4-dihydro-2H-benzo[b][1,4]oxazine-7-yl)sulfonyl)piperidine-3-carboxylate (0.56 g, 1.46 mmol) in tetrahydrofuran (10 mL). The reaction mixture was stirred at room temperature overnight, the solvent was evaporated, the residue was diluted with water and the solution was acidified to pH 1. The solution was extracted three times with ethyl acetate, and the combined organic layer was washed with 80% saturated brine, dried (magnesium sulfate), filtered and evaporated to give a product (92%).

¹H NMR (400 MHz, CDCl₃) δ 10.63 (br, 1H), 9.87 (s, 1H), 7.97 (s, 1H), 7.73 (s, 1H), 4.70 (q, J=6.8 Hz, 1H), 3.50 (m, 2H), 3.25 (m, 2H), 2.64 (s, 3H), 2.33 (m, 1H), 2.09-1.98 (m, 4H), 1.53 (m, 2H), 0.89 (m, 3H).

Mass spectrum: Found 383 [M+H]⁺, C17H22N2O6S requires 382

### 1-((2-Ethyl-6-methyl-3-oxo-3,4-dihydro-2H-benzo[b][1,4]oxazine-7-yl)sulfonyl)-N-(4-fluorophenyl)piperidine-3-carboxamide

A mixture of 1-((2-ethyl-6-methyl-3-oxo-3,4-dihydro-2H-benzo[b][1,4]oxazine-7-yl)sulfonyl)piperidine-3-carboxylic acid (0.15 g, 0.39 mmol), 4-fluoroaniline (0.065 g 0.59 mmol, 1.5 eq), bop reagent (259 mg, 0.59 mmol, 1.5 eq) and diethylisopropylamine (0.14 mL, 0.78 mmol, 2 eq) in THF (1 mL) was stirred at room temperature for 18 hours. The reaction mixture was poured into 5% NaHCO₃ (3 mL) and then stirred for 2 hours. The aqueous layer was decanted and the oily residue was dissolved in CH₂Cl₂ and purified by flash chromatography to give a product (20%).

¹H NMR (400 MHz, CDCl₃) δ 10.05 (s, 1H), 9.87 (s, 1H), 7.97 (s, 1H), 7.73 (s, 1H), 7.59 (m, 2H), 7.14 (m, 2H), 4.70 (q, J=6.8 Hz, 1H), 3.50 (m, 2H), 3.25 (m, 2H), 2.64 (s, 3H), 2.33 (m, 1H), 2.09-1.98 (m, 4H), 1.53 (m, 2H), 0.89 (m, 3H).

Mass spectrum: Found 476 [M+H]⁺, C23H26FN3O5S requires 475

### EXAMPLE 7

### Efficacy Analysis of Compound 1 and Compound 2

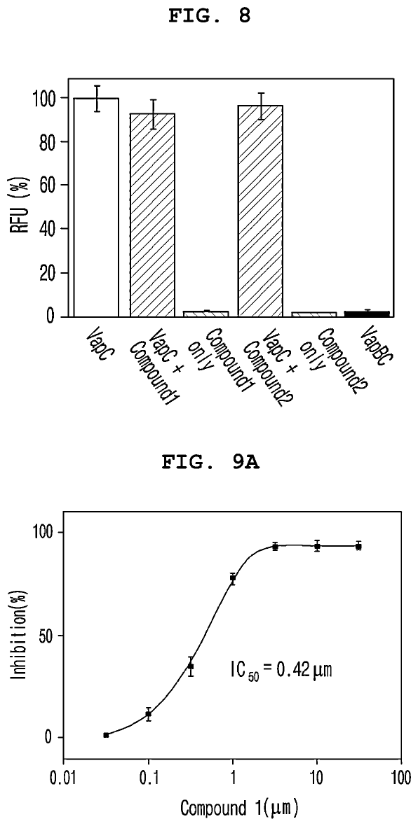

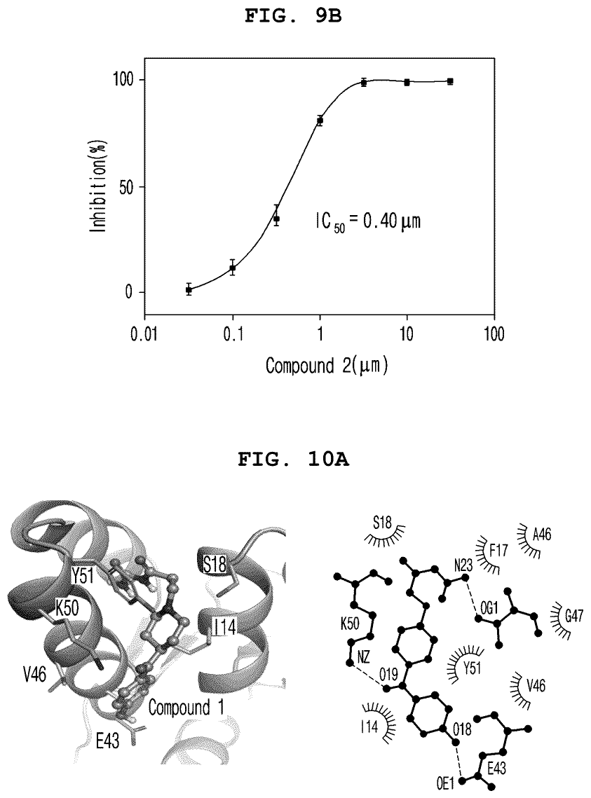

These two compounds showed higher efficacy at 1 μM, one hundredth of the peptide concentration used earlier (100 μM, Example 2). Therefore, the compounds 1 and 2 were very efficient in disrupting the VapBC complex. The compounds 1 and 2 showed IC₅₀ values of 0.42 and 0.40 μM, respectively (FIGS. 9A and 9B).

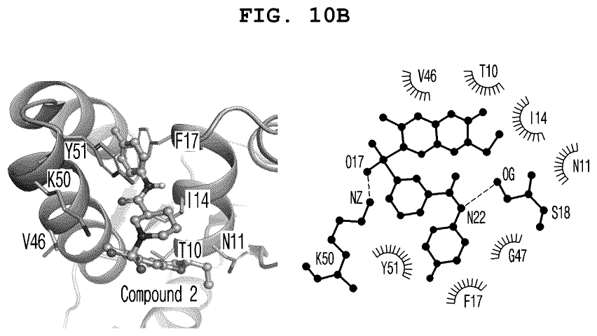

In addition, the molecular docking simulation results of the compounds 1 and 2 are provided in FIGS. 10A and 10B, respectively. The carbonyl group of the compound 1 interacted with the positively charged amino group in the side chain of K50 by forming strong HB. The residues E43, S18 and A48 also formed carbon-hydrogen bond interactions with hydrogen. The residues Y51, I14 and V46 formed π-alkyl and alkyl interactions. Similar to the HB of the compound 1, the sulfonyl group of the compound 2 was also found to have a positively charged amino group and strong HB in the side chain of K50. In addition, the residues T10, N11, G47 and A48 formed carbon-hydrogen bonds and amide-n stacking interactions. The residues I14 and V46 were involved in the formation of π-alkyl and alkyl interactions, respectively. F17 and Y51 formed π-π stacking interactions with the fluorobenzene ring of the compound.

From the above results, it was confirmed that the compounds 1 and 2 are small molecules that control the bacterial TA system and can control drug-resistant Klebsiella pneumoniae.

### EXAMPLE 8

### Potential Analysis of Competitive Inhibitors and Autoregulation Based on Binding Affinity

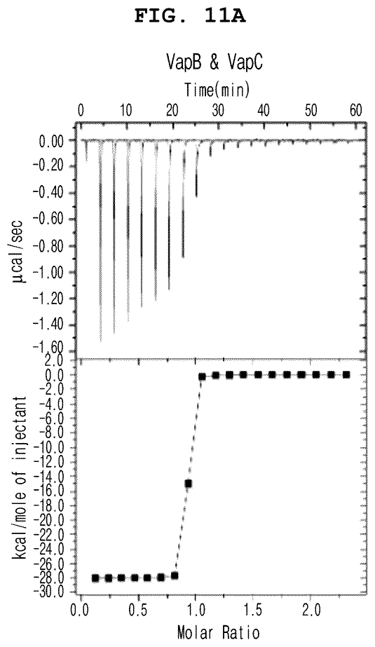

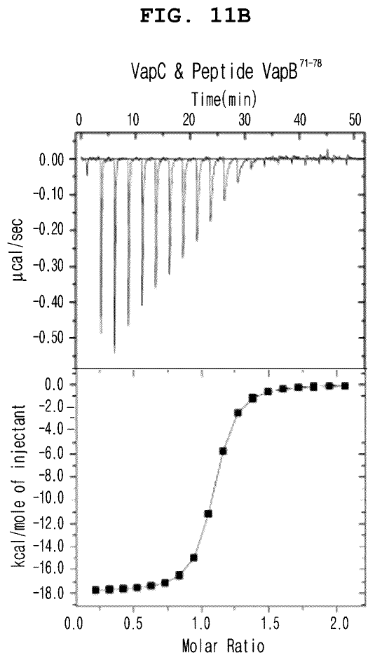

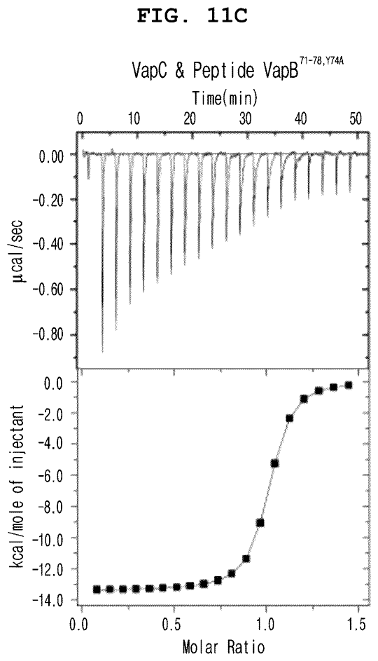

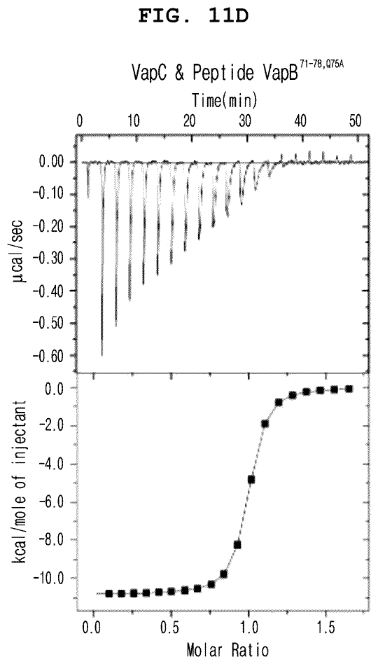

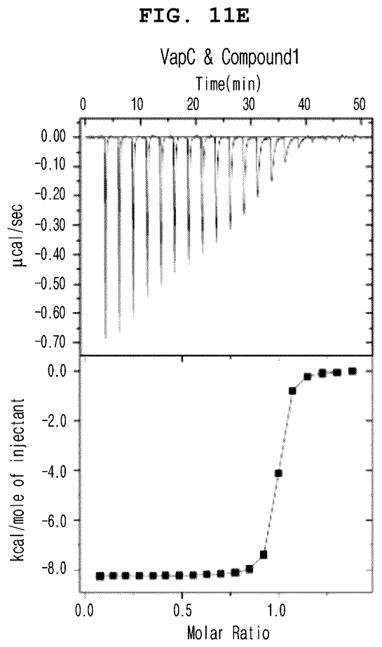

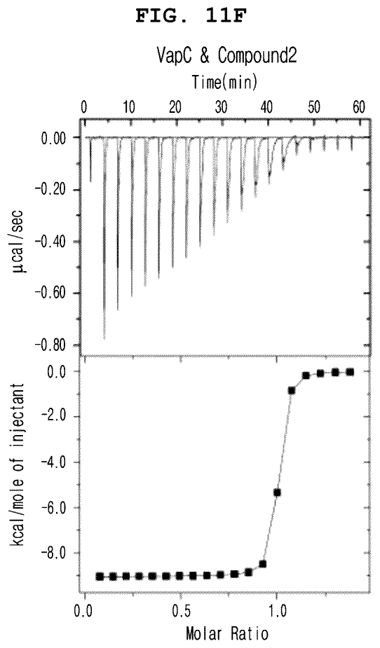

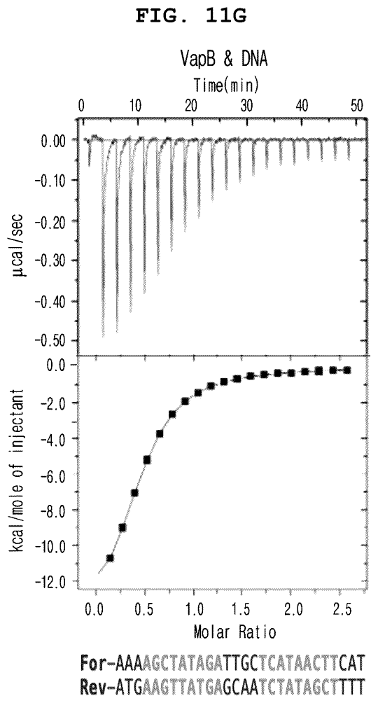

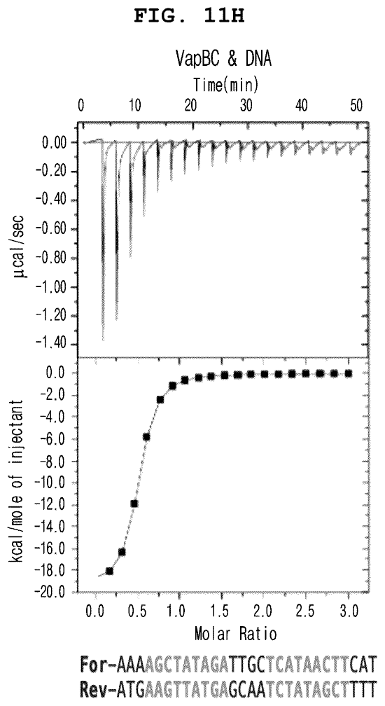

To further verify the peptides and small molecules selected in the present invention, the binding affinities of VapC and VapB, peptides and small molecules were measured by isothermal titration calorimetry (ITC) (FIGS. 11A-11H).

ITC was performed using MicroCal200 (GE Healthcare). The concentrations of protein and DNA were determined using NanoDrop (Thermo) at 280 nm and 260 nm, respectively. Stock solutions of peptides and small molecules were prepared at appropriate concentrations. Titration was performed at 25° C. The data were calculated by subtracting the background and fitted to a single-site binding model using the provided source-based software with MicroCal200. The results are shown in FIG. 11 and Table 5.

TABLE 5

| | n (the binding | K_{d} | ΔH | TΔS | ΔG |
|---|---|---|---|---|---|
| | stoichiometry) | (μM) | (kcal mol⁻¹) | (kcal mol⁻¹) | (kcal mol⁻¹) |
| VapB & VapC | 0.97 ± 0.002 | 0.003 ± 0.0002 | −28.1 ± 0.04 | −16.4 | −44.5 |
| VapC & Peptide VapB⁷¹⁻⁷⁸ | 1.02 ± 0.002 | 0.11 ± 0.0002 | −17.9 ± 0.07 | −8.4 | −26.3 |
| VapC & Peptide VapB^{71-78, Y74A} | 0.99 ± 0.001 | 0.17 ± 0.0002 | −13.4 ± 0.02 | −4.17 | −17.57 |
| VapC & Peptide VapB^{71-78, Q75A} | 0.99 ± 0.001 | 0.15 ± 0.0003 | −10.8 ± 0.02 | −1.53 | −12.33 |
| VapC & Compound 1 | 0.98 ± 0.002 | 0.035 ± 0.0004 | −8.3 ± 0.03 | 1.93 | −6.37 |
| VapC & Compound 2 | 0.99 ± 0.004 | 0.022 ± 0.0002 | −9.1 ± 0.06 | 1.39 | −7.71 |
| VapB & DNA | 0.48 ± 0.002 | 5.38 ± 0.0002 | −15.3 ± 0.05 | −8.08 | −23.38 |
| VapBC & DNA | 0.49 ± 0.003 | 1.26 ± 0.0004 | −19.54 ± 0.03 | −11.51 | −31.05 |

VapB was most strongly bound with VapC (K_{d}=3 nM), and the peptides and small molecules showed affinity with VapC in the nanomolar range (peptides: K_{d}=110-170 nM, small molecules: K_{d}=22-35 nM) (FIG. 11). This confirmation of affinity confirms that the peptides were successfully reasonably designed as competitive inhibitors for the VapB binding region, and that the proposed docking model of the small molecules is reliable.

The affinities of the peptides VapB⁷¹⁻⁷⁸, VapB^{71-18, Y74A}, and VapB^{71-78, Q75A} to VapC were 110 nM, 170 nM, and 150 nM, respectively. Mutations in Y74 and Q75 caused some of the interactions with VapC to disappear and the affinity with VapC to decrease. However, Y74 and Q75 of VapB interacted with the active site of VapC. Therefore, VapB^{71-78, Y74A} and VapB^{71-78, Q75A} exhibited higher activity than VapB⁷¹⁻⁷⁸ due to a decrease in VapC activity inhibition, despite a decrease in binding affinity with VapC.

In the type II TA system, antitoxins and toxin proteins play an important role in transcriptional autoregulation. To investigate the molecular mechanism of transcriptional regulation, a 28-bp DNA sequence in the promoter region was titrated with VapB and VapBC. The affinity of VapBC to DNA (K_{d}=1.26 μM) was five times higher than that of VapB to DNA (K_{d}=5.38 μM). This indicates that VapB acts as a major repressor in autoregulation by binding directly to its own promoter, whereas VapC acts as a corepressor that forms a complex with VapB to strengthen the interaction between VapB and the operator site.

### Conclusion

The structure of the VapBC complex of the present invention is the first TA complex structure reported in Klebsiella pneumoniae. The mechanism of VapC toxin suppression by R79 of VapB replacing Mg²⁺ has been revealed through two crystalline structures, the open and closed forms of the active site. This is a novel invention confirmed for the first time in the field of bacterial TA systems. Through the rational design of peptides mimicking the VapBC binding interface, the VapBC complex can be disrupted and artificial activation of the toxin can be applied to Klebsiella pneumoniae as an antibacterial strategy. In addition, by providing small molecule compounds that effectively inhibit the formation of VapBC complexes even at low concentrations, the present invention can enable the control of drug-resistant Klebsiella pneumoniae through modification with inhibitors.

1. A peptide that inhibits the binding of VapB and VapC in a Klebsiella pneumoniae toxin-antitoxin complex VapBC.

2. The peptide according to claim 1, wherein the peptide has any one amino acid sequence selected from the group consisting of the sequences represented by SEQ ID NO: 17 TO SEQ ID NO: 26.

3. The peptide according to claim 1, wherein the peptide inhibits the binding of an α3 helix of the VapB and an α1 helix of the VapC.

4. The peptide according to claim 1, wherein the peptide exhibits antibacterial activity against Klebsiella pneumoniae.

5. A compound that inhibits the binding of VapB and VapC in a Klebsiella pneumoniae toxin-antitoxin complex VapBC.

6. The compound according to claim 5, wherein the compound is compound 1 or 2 below:

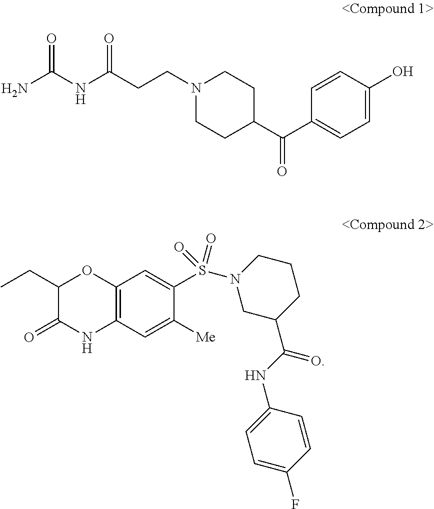 [SMILES: NC(=O)NC(=O)CCN1CCC(C(=O)c2ccc(O)cc2)CC1 ; CCC1Oc2cc(S(=O)(=O)N3CCCC(C(=O)Nc4ccc(F)cc4)C3)c(C)cc2NC1=O]

7. The compound according to claim 5, wherein the compound exhibits antibacterial activity against Klebsiella pneumoniae.

8. An antibacterial pharmaceutical composition comprising the peptide of claim 1.

9. The antibacterial pharmaceutical composition according to claim 8, wherein the composition exhibits antibacterial activity against Klebsiella pneumoniae.

10. The antibacterial pharmaceutical composition according to claim 8, wherein the composition is effective to prevent or treat pneumonia.

11. An antibacterial quasi-drug comprising the peptide of claim 1.

12. An antibacterial external preparation comprising the peptide of claim 1.

13. An antibacterial pharmaceutical composition comprising the compound of claim 5.

14. The antibacterial pharmaceutical composition according to claim 13, wherein the composition exhibits antibacterial activity against Klebsiella pneumoniae

15. The antibacterial pharmaceutical composition according to claim 13, wherein the composition is effective to prevent or treat pneumonia.

16. An antibacterial quasi-drug comprising the compound of claim 5.

17. An antibacterial external preparation comprising the compound of claim 5.
